(19) United States
(12) Patent Application Publication
(10) Pub. No.: US 2003/0109923 A1
(43) Pub. Date: Jun. 12, 2003

(54) Polymer heart valve with perforated stent and sewing cuff

(76) Inventors: Joseph A. Chinn, Austin, TX (US); Riyad Moe, Austin, TX (US); James Charles Caffey, Marble Falls, TX (US)

Correspondence Address: SULZER MEDICA USA INC., Suite 1600, 3 East Greenway Plaza, Houston, TX, 77046, US

(21) Appl. No.: 10/133,859

(22) Filed: Apr. 27, 2002

Related U.S. Application Data
(63) Continuation-in-part of application No. 10/020,337, filed on Dec. 12, 2001.

(51) Int. Cl. A61F 2/24

(52) U.S. Cl. 623/2.17; 623/2.4

(57) ABSTRACT

A prosthetic heart valve having an elastomeric valve body with an annular base for receiving a suture. A sewing cuff is coupled to the valve body and/or a stent member by a piercing suture or other connector member, such as a staple or pin. Apertures may be provided in the stent member.

### BACKGROUND

The present application is a continuation-in-part of co-pending U.S. patent application Ser. No. 10/020,337, filed Dec. 12, 2001, which is specifically incorporated by reference herein in its entirety.

### TECHNICAL FIELD

The present invention pertains to prosthetic heart valves and in particular to polymeric tri-leaflet heart valve prostheses.

### BACKGROUND OF THE INVENTION

It has been possible since 1950, when blood oxygenators made open heart surgery feasible, to treat some forms of heart disease by replacing a patient's heart native heart valve with a prosthetic valve. The prosthetic heart valve is implanted into an annular opening in a heart created when the diseased valve is removed. Early heart valve prostheses included ball-and-cage valves and disc-and-cage valves in which a ball or a disc was housed in a cage. In these valves, one side of the cage provides an orifice through which blood flows. When blood flows in a forward direction, the energy of the blood flow forces the ball or disc to the back of the cage, allowing blood to flow through the valve. When pressure reverses and the blood begins to flow in a reverse direction, or “regurgitate,” the energy of the blood flow forces the ball or disc into the orifice of the valve, blocking flow in the reverse direction. In this way, the valve functions as a one-way check valve for blood flow.

Ball-and-cage and disc-and-cage valves are examples of the class of “mechanical” heart valve prostheses. More recent examples of mechanical valves include bileaflet and tilting disc valves having pivoting and/or sliding leaflet occluders for regulating blood flow (hereinafter referred to interchangeably simply as “leaflets”). Mechanical valves are characterized by rigid or semi-rigid leaflets which operate by movement (i.e., pivotal movment and/or translational movement) between an open position allowing blood flow in a forward direction and a closed position preventing blood flow in a reverse direction. The energy of blood flow causes the occluders to move between their open and closed positions. Mechanical valves typically comprise an annular valve body having one, two, or three rigid leaflet occluders pivotally coupled to the valve body.

More recently, “tissue valves” comprising at least some components made of tissue or tissue products have been used as heart valve prostheses. The valve leaflets of tissue valves are usually flexible and made from tissue, such as specially treated porcine or bovine pericardial tissue. A tri-leaflet tissue valve may comprise an annular valve body in which three flexible leaflets are coupled to a supporting portion of the valve body, called a “stent,” located at the circumference of the annulus. Other, so-called “stentless” designs avoid the use of a supporting structural stent member and typically comprise only tissue or tissue-derived components coupled together. When blood flows in the forward direction, the energy of the blood flow deflects the three leaflets away from the center of the annulus and allows blood to flow through. When blood flows in the reverse direction, the three leaflets engage each other in a coaptive region, occlude the valve body annulus and prevent the flow of blood.

“Polymer valves” comprise a third type of prosthetic heart valve, in which at least the leaflets of the valve are made from a man-made elastomeric polymer such as polyurethane or another biocompatible polymer. Polymer valves typically comprise a valve body, which may also comprise a polymer, and two or three leaflets. A stent may also be provided to increase the structural strength of the valve body while allowing the leaflets to remain flexible. Polymer valves may be sutured or pinned directly to the site of an explanted heart valve, or a sewing cuff (interchangeably referred to herein as “suture ring”) may be coupled to the valve body. The valve may then be attached to the heart by suturing the sewing ring to the heart annulus of the patient.

Where the valve body comprises a polymer, care must be taken in how the sewing cuff is coupled to the polymeric body to avoid placing undesired stress on the valve body or leaflets. Prior art approaches have avoided sutures which pierce the valve body to reduce the risk of initiating cracks or stresses in the polymer, which can lead to premature valve failure. In one prior art approach, the sewing cuff is compressibly retained against the valve body by a suture, wire, or elastomeric belt wrapped around the outer periphery of the suture ring a number of times. In copending U.S. patent application Ser. No. 10/020,337, a number of approaches are provided for coupling the sewing cuff to the valve body without penetrating the elastomeric components of the valve.

Consistent with the foregoing, prior art approaches to prosthetic heart valves have focused on the need for long term (i.e., years or decades) operability and robustness. However, recent advances in heart assist devices, include artificial heart devices and left ventricular assist devices, have created a need for valves that are biocompatible, inexpensive, and durable, but which are implanted for weeks or months rather than years. Polymer heart valves are expecially well-suited to such uses, because they can be made using inexpensive, highly repeatable processes with extremely precise tolerances such as molding. The present inventors have discovered that polymer valves can be fabricated that retain all or most of the foregoing advantages, but can be made at lower cost, by providing a simpler method of attaching the suture cuff to the valve body.

### SUMMARY OF THE INVENTION

In one aspect, the invention provides a polymer heart valve having a polymeric, annular valve body and a plurality of flexible polymeric leaflets coupled to the body. In a preferred embodiment, both the valve body and leaflets comprise an elastomeric polymer. The valve body preferably comprises an upper (or downstream) end having a plurality of posts joined by attachment curves extending between the posts. Each leaflet is preferably coupled to a corresponding attachment curve. The valve body further preferably comprises a lower (or downstream) end to which the sewing cuff is coupled by one or more sutures penetrating both the polymeric valve body and the sewing cuff. As used herein, the term “pass through,” or any form thereof, refers to an instance where a suture or other connnection means enters and exits an opening substantially larger than the diameter of the suture or connecting means. The terms “pierce” and “penetrate,” or any form thereof, refer to an instance where a suture or other connection means enters and exits an opening substantially the same as, or only slightly larger than, the diameter of the suture or connecting means, as for example a suture pulled through an opening created by a needle coupled to the suture.

The valve according to the present invention may optionally comprise a stent coupled to the valve body to provide additional support for the valve body. Where a stent if provided, the suture may optionally pierce the stent in addition to the valve body and the sewing cuff. Alternatively, the stent may be provided with apertures such that the suture pierces only the valve body and sewing cuff, while passing through the apeartures in the stent. In another alternate embodiment, the stent may extend below the base of the valve body, and the sewing cuff may be sutured directly to (i.e., may pierce) the stent and cuff without piercing or passing through the valve body. In a further embodiment, the valve may comprise a stent that does not extend to the base of the valve body, thus enabling the sewing cuff to be sutured to the valve body by piercing the valve body and cuff, without passing through or penetrating the stent.

Co-pending U.S. patent application Ser. No. 10/020,337 discloses heart valve prostheses, including polymer heart valves, in which the sewing cuff is coupled to the valve body without the need to pierce the polymeric (or tissue) components of the valve. While such approaches potentially can improve the durability of polymer heart valves, there is nevertheless an increase in the complexity of the design and manufacturing processes for the valve. In contrast to this approach, the present inventors have developed a much simpler and less expensive method of coupling the sewing cuff to the valve. Valves according to the present inventions may be rapidly assembled without complex machinery.

In another aspect, the invention comprises a method of attaching a sewing cuff to a polymeric valve body. The method may comprise suturing a sewing cuff to the polymeric valve body by piercing the valve body and cuff. In an alternative embodiment, the method may comprise suturing the sewing cuff to the valve body and to a stent member coupled to the valve body, piercing the sewing cuff, the valve body and the stent. In a further embodiment, the method may comprise suturing the sewing cuff to the stent member and not to the valve body. In this embodiment, the suture (or other connection means such as a staple) pierces the stent and the cuff and does not pierce or pass through the valve body. It is an object, therefore, of the present invention to provide a polymeric prosthetic heart valve with means for inexpensively and rapidly attaching a sewing cuff to the valve. These and other objects and features of the invention will be apparent from the following detailed description, made with reference to the accompanying drawings.

### BRIEF DESCRIPTION OF DRAWINGS

FIG. 1 is a perspective view of a polymer heart valve.

FIG. 2 is a perspective view of a stent for a polymeric heart valve.

FIG. 3 is a perspective view of a portion of a further embodiment of the stent of Fig. 2.

FIG. 4 is a through section of a portion of the stent of FIG. 2, taken at line IV-IV.

FIG. 5 is a perspective view of the stent of FIG. 2 with elastomeric polymer leaflets molded thereon.

FIG. 6 is a through section of a portion of the stent and leaflets of FIG. 5 taken at line V-V.

FIG. 7 is the view of FIG. 6 with a molding pin inserted through an aperture in the stent.

FIG. 8 is a perspective view of a polymeric heart valve with stent and leaflets as shown in FIG. 5 and attached sewing cuff.

FIG. 9 is a partial through section of a portion of a heart valve illustrating attachment of a sewing cuff.

FIG. 10 is a through section similar to FIG. 6, illustrating a partially enclosed stent.

FIG. 11 is the view of FIG. 10, further illustrating an attached sewing cuff. FIG. 12 is a view of a portion of a further embodiment of a stent for use in a polymer heart valve.

FIG. 13 is a through section view of the stent of FIG. 12 at line XII-XII, further illustrating an attached sewing cuff.

FIG. 14 is a partial through section of a portion of a stentless heart valve illustrating attachment of a sewing cuff to a polymeric valve body by sutures, with the suture passing through the valve body and the sewing cuff.

FIG. 15 is a partial through section of a portion of a stented heart valve illustrating attachment of a sewing cuff to a polymeric valve body by sutures, with the suture passing through the valve body and the sewing cuff, but not the stent member.

FIG. 16 is a partial through section of a portion of a stented heart valve illustrating attachment of a sewing cuff to a polymeric valve body and to a stent member by sutures, with the suture passing through the valve body, the stent member, and the sewing cuff.

FIG. 17 is a partial through section of a portion of a stented valve having apertures in the stent member, illustrating attachment of a sewing cuff to a polymeric valve body, with the sutures passing through the valve body, an aperture in the stent member, and the sewing cuff.

FIG. 18 is a partial through section of a portion of a stented polymer valve illustrating attachment of a sewing cuff to the valve, with the suture passing through the stent member and the sewing cuff, but not through the polymer valve body.

### DESCRIPTION OF THE PREFERRED EMBODIMENTS

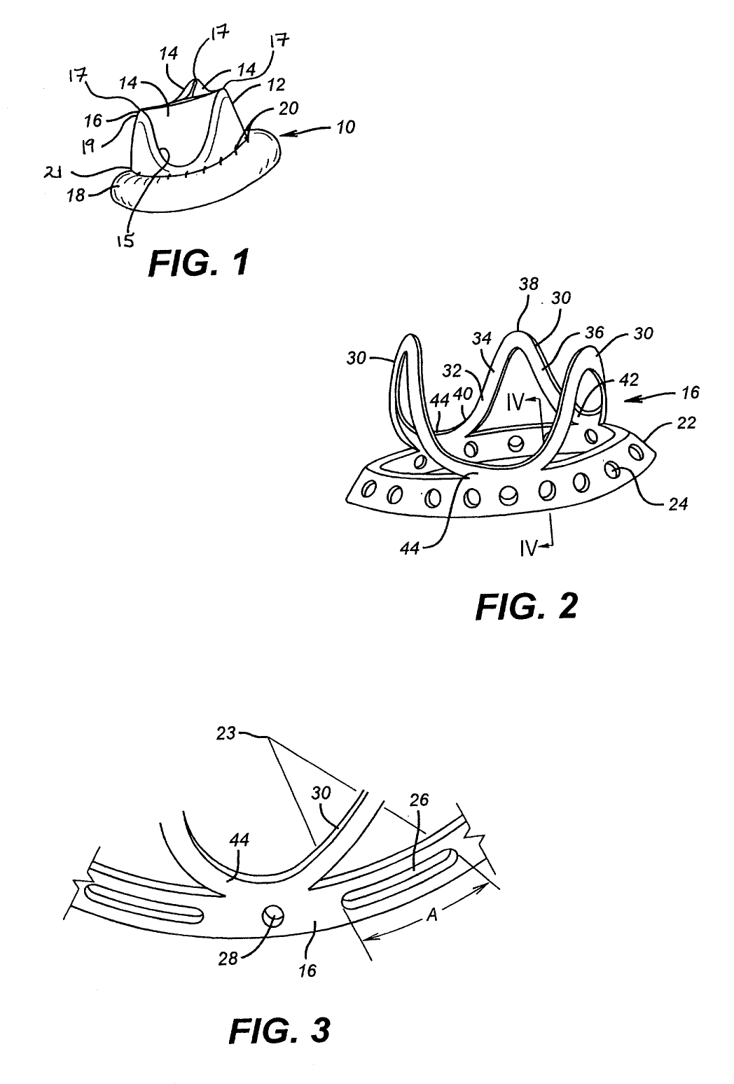

A tri-leaflet heart valve 10 comprises an annular elastic valve body 12 having an upper, downstream end 19, a lower, upstream end 21, and three flexible leaflets 14 made of a biocompatible polymer such as silicone or polyurethane, as shown in FIG. 1. A stent 16, made of metal or plastic, reinforces the elastic valve body. The stent 16 is at least partially embedded in the elastic material that forms the valve body 12. A sewing cuff 18 is coupled by, e.g., sutures 20 to the lower end 21 of valve body 12. Other attachment means such as pins or staples may also be used. In certain embodiments, the pins or sutures pierce the sewing cuff but pass through apertures in the stent and elastic material as depicted in FIGS. 8, 9, 11, and 13. In other embodiments, the pins or sutures pierce the valve body 12 and/or stent member 16 in addition to the sewing cuff, as depicted in FIGS. 14-18. The valve 10 may also omit the sewing ring 18.

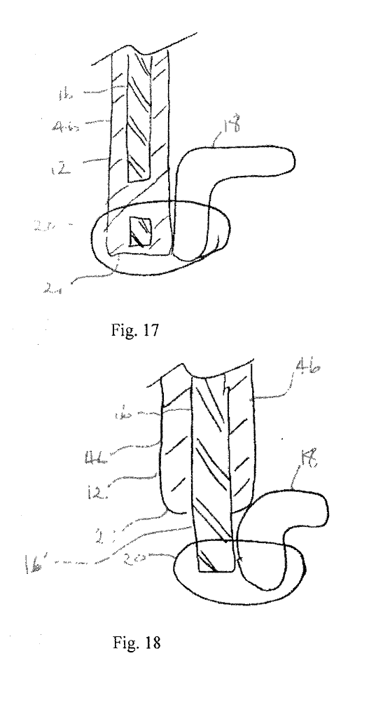

In FIG. 2, a stent 16 is provided having a circumferential base 22. The base may be generally ring-like in configuration or it may be scalloped to conform to the anatomy common at sites of explanted natural valves. In one embodiment, a plurality of apertures or holes 24 spaced circumferentially around the base provides openings for sutures, pins, or other attachment apparatus, as explained below. The apertures 24 may have any suitable shape, such as round, elliptical or elongated, such as slots 26 as depicted in FIG. 3. In some embodiments, the apertures are open, as depicted in e.g., FIGS. 9, 11, and 13, thus allowing a connecting means to pass through the apertures. Alternatively, the apertures may be embedded, along with other portions of the stent, in the polymeric valve body, as depicted in FIG. 17, in which case the connecting means pierces the valve body while passing through the apertures. Sutures can be threaded through the slots 26 and the valve may be rotated into a slightly more favorable orientation. Other round apertures 28 may be used to secure the valve in a final position. In still another alternative embodiment, the stent 16 can be provided having no apertures, as shown in FIGS. 15, 16, and 18.

Returning to the stent of FIG. 2, a plurality of commissure supports 30 rise from the base 22 and define a generally cylindrical area for the leaflets 14. FIG. 2 depicts three such supports for a tri-leaflet valve; a bi-leaflet valve would have two commissure supports. More leaflets and commissures are possible, but are generally not justified because of increased complexity. In a preferred embodiment, a commissure support 30 comprises a ribbon-like segment of polymeric or other material formed into two opposed concave legs 34, 36. The legs 34, 36 are flared apart near the base 22. Distally from the base 22, the legs 34, 36 approach each other and are joined by a convex apex segment. Each leg 34,36 is joined to the base 22 at a proximal end 40, 42. Preferably the commissure supports 30 taken together form a smooth, closed curve circumferentially around the base 22. The apex segments 38 usually form tighter curves than a curve 44 between two adjacent commissure supports. Those of skill in the art will recognize that other configurations and materials, such as wire, for example, may be used for commissure supports.

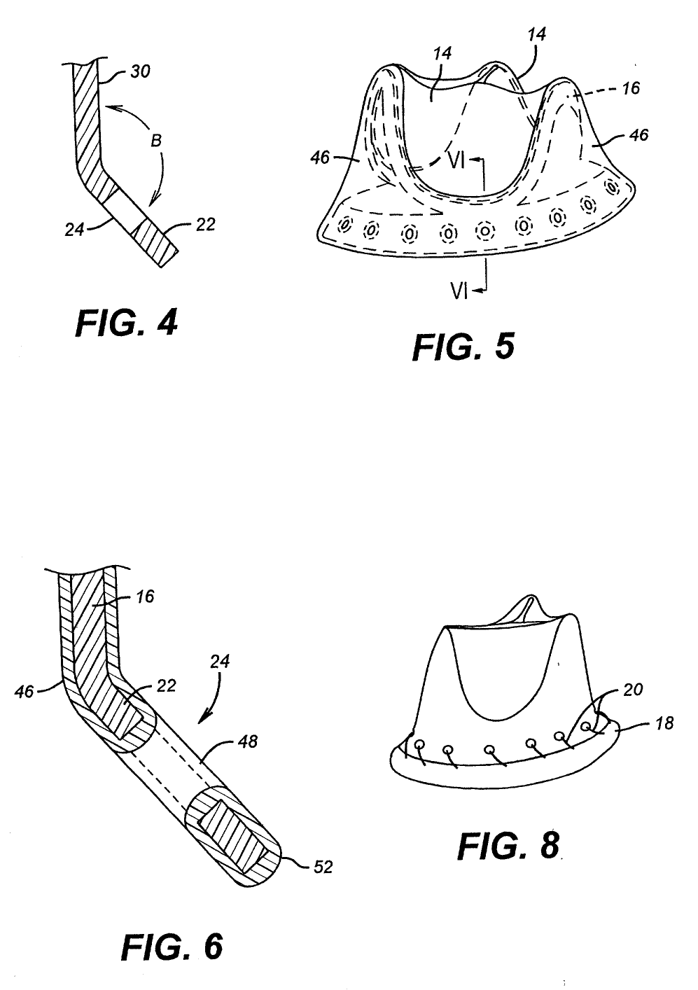

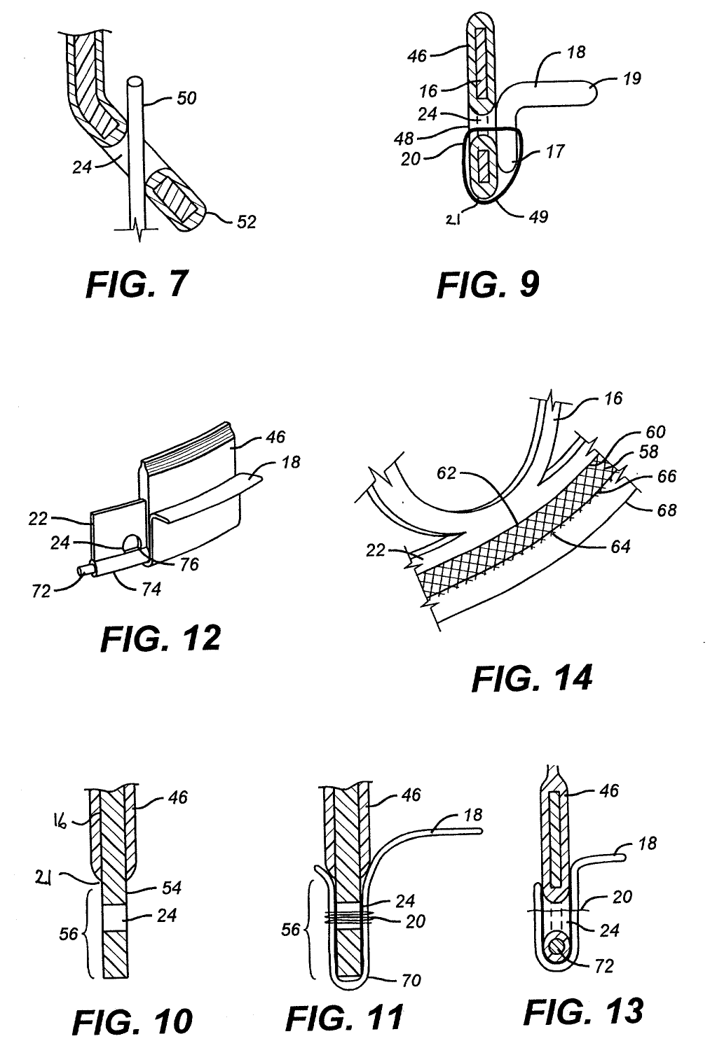

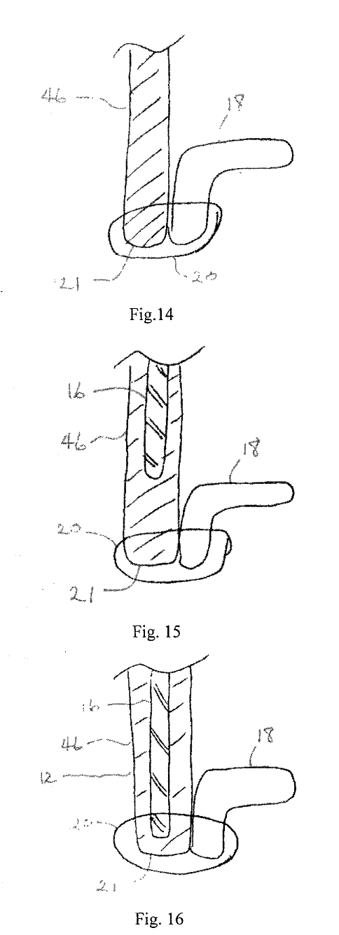

In certain embodiments, the base 22 may be flared outwardly away from the commissure supports, as shown in FIG. 2. The base has a frustro-conical shape and forms an angle B as shown in FIG. 4 between the commissure supports 30 and the base 22. The angle B is preferably between about 90° and about 130°. This angle helps in manufacturing certain configurations of heart valves. In certain configurations, the base may be cylindrical, as shown in FIGS. 10 through 18.

The stent 16 may be placed in a mold and encased or embedded in a flexible polymeric material 46, as shown in FIG. 5. FIG. 5 depicts the stent completely embedded within the polymeric material. Alternate embodiments provide a valve in which the stent is only partially embedded in the polymeric material, as shown in FIGS. 10, 11, and 18. The flexible polymer material is simultaneously formed into leaflets 14. In one embodiment, the polymeric material 46 is formed over the stent 16 so as to leave an opening 48 though the aperture 24. This may be done, for instance, by inserting a pin 50 through the aperture 24, as shown in FIG. 7. In a preferred embodiment, the polymeric material 46 is formed over the stent 16 so as to embed the aperture 24 within the polymeric material, as shown in FIG. 17.

One way of attaching the sewing cuff to a valve body is depicted in FIG. 9. Sewing cuff 18 is placed against the outside of the valve body and sutures 20 or other fasteners are passed through the opening 48 in the aperture 24. The suture then pierces the sewing ring, then passes beneath lower edge 21 of the valve body. The suture material may then either be tied in a loop or passed again through the valve body opening and pierce the sewing cuff to form additional loops through the valve body. The sutures or fasteners attach a secured end 17 of the sewing ring to the annular valve body. A free end 19 of the sewing ring extends radially outwardly, forming a cuff area that can receive sutures to attach the valve to cardiac tissue.

Yet another method of attaching a sewing ring to a polymer valve body is illustrated in FIG. 14. In the embodiment of FIG. 14, however, in contrast to the embodiment of FIG. 9, the connector means pierces the polymeric material 46 that comprising the valve body 12. More specifically, sewing cuff 18 is placed against the outside of valve body 12 and sutures 20 or other fasteners then pierce the flexible polymeric material 46 of valve body 12, pierce the sewing cuff, and pass beneath lower edge 21 of the valve body to form either a single loop or by repeated piercing-and-wrapping steps form a number of loops from a single strand of suture material. A secured first end 17 of the sewing ring is attached to polymeric material 46, and a second, free end 19 extends radially outward, forming a cuff that can receive sutures to attach the valve to the patient's heart tissue.

In contrast to the stentless embodiment of FIG. 14, valve 10 may also comprise a stent 16, as depicted in FIGS. 15 and 16. The stent of FIG. 15, while embedded in polymeric material 46, does not extend to the lower edge 21 of the valve. Accordingly, in the embodiment of FIG. 15, the suture 20 pierces valve body 12 and cuff 18 but does not pierce stent 16. The embodiment depicted in FIG. 16, by contrast, comprises a stent 16 that extends to a location proximate lower edge 21 of the valve. In this embodiment, the suture 20 pierces polymeric material 46 of valve body 12, stent 16 and sewing cuff 18.

FIG. 18 illustrates a still further embodiment of the present invention. A stent 16 extends below (or upstream) of the lower edge 21 of valve body 12. Thus, a portion 16′ of stent 16 is exposed instead of embedded in polymeric material 46. The sewing cuff 18 may be directly coupled by suture 20 to stent 16, piercing the exposed portion 16′ of stent 16 and cuff 18, but not piercing valve body 12.

A related embodiment to that of FIG. 18 is provided in FIGS. 10 and 11. There, a series of suture apertures or holes free from polymeric material are provided by only partially covering the base with polymeric material. As illustrated in FIG. 10, the polymeric material 46 extends only to a midpoint 54 of the base, leaving an exposed region 56. Campbell and Moe have suggested partially exposing a linear portion of a stent in commonly assigned U.S. application Ser. No. 09/174,387, incorporated herein in its entirety by this reference. As shown in FIG. 11, the sewing cuff 18 comprises an attachment portion 70 folded to contact both the inside and outside surfaces of the exposed region 56. The suture 20 is passed through the apertures 24 and pierces the attachment portion of the cuff on both the inside of the attachment portion and the outside of the attachment portion. Since the polymeric material 46 does not reach the apertures, the structural integrity of the polymeric material is not compromised by a needle drawing the suture through the apertures 24 or by another fastener inserted through the apertures.

The embodiment of FIG. 12 and FIG. 13 is similar is to the embodiment of FIG. 10 and FIG. 11. A wire 72 is cast into the base 22 of the stent 16 at an upstream edge 74. The stent is coated with polymeric material 46 and the sewing ring 18 is attached by passing the suture 20 through the apertures 24 and piercing the cuff. The imbedded wire 72 gives additional strength to the base to resist forces acting on the valve in the downstream or forward direction of blood flow through the valve. Because of this added strength, the apertures 24 can be placed off-center on the base 22. With the apertures closer to the upstream edge 74, more of the material in the base 22 can resist forces acting on the valve in the upstream direction, that is when the valve closes. Consequently, the overall height of the base can be reduced. Apertures may be formed as a plurality of notches 76 along the upstream edge 74 of the base of the stent with the circumferential wire 72 lying along the upstream edge 74 of the base and closing an open side of the notches. The sewing cuff 18 can be attached either from the outside, as in FIG. 9, by folding an attachment portion on both the inside and the outside of the base 22, as shown in FIG. 13, or from the inside, as shown in FIG. 8. The base 22 may be canted outwardly to form a frustro-conical ring, as described above in connection with FIG. 2.

The foregoing describes preferred embodiments of the invention and is given by way of example only. For example, the invention is not limited to the manufacturing techniques disclosed but includes any manufacturing technique that leaves a portion of the stent outside the elastic material of the valve body. Further, the invention includes any prosthetic valve in which the prosthetic valve can be implanted without sutures or pins or the like piercing the elastic material of the valve body. The invention is not limited to any of the specific features described herein, but includes all variations thereof within the scope of the appended claims.

What is claimed is:

1. A prosthetic heart valve comprising:
a polymeric valve body having at least one leaflet, said leaflet having an open position and a closed position,
a sewing cuff coupled to said valve body by a suture, said suture piercing said polymeric valve body and said sewing cuff.

2. The prosthetic heart valve of claim 1 wherein said prosthetic heart valve further comprises a stent coupled to said valve body.

3. The prosthetic heart valve of claim 2 wherein said suture pierces said stent in addition to piercing said valve body and sewing cuff.

4. The prosthetic heart valve of claim 2 wherein said stent comprises a plurality of apertures and wherein said suture does not pierce said apertures.

5. The prosthetic heart valve of claim 2 wherein said suture does not pierce said stent.

6. The prosthetic heart valve of claim 1 wherein said valve does not comprise a stent.

7. A prosthetic heart valve comprising:
a polymeric valve body having at least one leaflet, said leaflet having an open position and a closed position,
a stent, and
a sewing cuff coupled to said stent by a suture, said suture piercing said stent and said sewing cuff.

8. The prosthetic heart valve of claim 7 wherein said stent comprises an exposed portion not embedded in said valve body and said suture does not pierce said valve body.

9. The prosthetic heart valve of claim 7 wherein said suture pierces said valve body.

10. A method of assembling a prosthetic heart valve comprising:
providing a polymeric valve body having at least one leaflet, said leaflet having an open position and a closed position,
providing a sewing cuff, and
coupling said sewing cuff to said valve body by
providing a suture having first and second ends,
piercing said polymeric valve body with said suture,
piercing said sewing cuff with said suture, and
forming a loop by coupling said first and second ends of said suture.

11. The method of claim 11, further comprising the step of providing a stent member prior to said step of coupling the sewing cuff to the valve body.

12. The method of claim 11, wherein said step of coupling the sewing cuff to the valve body further comprises the step of piercing said stent prior to said step of forming a loop.
